(19) United States
(12) Patent Application Publication
(10) Pub. No.: US 2023/0117459 A1
(43) Pub. Date: Apr. 20, 2023

(54) POWDERY COMPOSITION FOR PRODUCING A DRINKABLE PRODUCT

(71) Applicant: PM-INTERNATIONAL AG, Schengen (LU)

(72) Inventors: Rolf Sorg, Schengen (LU); Tobias Kühne, Morbach (DE); Wihelm Messer, Bad Dürkheim (DE)

(21) Appl. No.: 17/910,219

(22) PCT Filed: Jan. 14, 2021
(86) PCT No.: PCT/EP2021/050683

§ 371 Date: Sep. 8, 2022

(30) Foreign Application Priority Data
Mar. 11, 2020 (DE) 10 2020 106 691.0

(51) Int. Cl. A23L 2/395; A23L 33/105; A23L 33/125; A23L 33/155

(52) U.S. Cl. CPC A23L 2/395; A23L 33/105; A23L 33/125; A23L 33/155

(57) ABSTRACT

A powdered composition for the preparation of a solution for oral administration, wherein the composition contains, per 100 g, 1 g to 85 g of at least one monosaccharide, 0.18 g to 0.70 g of at least one steviol glycoside, 1.5 g to 10 g of citric acid, 0.3 g to 3 g of vitamin C, at least two other vitamins, and an extract from green tea (Camellia sinensis).

### CROSS-REFERENCE TO RELATED APPLICATIONS

The present application is a national stage application filed under 35 U.S.C. § 371 of International Application No. PCT/EP2021/050683, filed Jan. 14, 2021, which claims priority to German Patent Application No. 10 2020 106 691.0, filed Mar. 11, 2020. Each of the aforementioned Patent Applications is incorporated herein by reference in its entirety for all purposes.

The invention relates to nutritional supplements, in particular to a powdered composition for the preparation of a solution for oral administration, and to the use of the inventive composition for the preparation of a solution for oral administration.

Nutritional supplements are usually administered orally. To improve the absorption of valuable ingredients in the gastrointestinal tract, to simplify the ingestion of the nutritional supplement and to avoid complications during ingestion, these nutritional supplements are often taken as an aqueous solution. To reduce microbial infestation and increase chemical stability, effervescent tablets or powders are commonly offered for this purpose, which are then dispersed in water by the user to produce a drinkable solution.

Inadequate in many cases is the dissolution behavior of the ingredients, the visual appearance and the taste of the solution. Also unsatisfactory in many nutritional supplements is the insufficient bioavailability of valuable ingredients. In general, the chemical and microbial stability of conventional nutritional supplements can also be improved. Thus, there is a need for improved nutritional supplements.

Nutritional supplements are also used to improve the supply of muscle and nerve cells. A good oxygen supply is considered essential, especially for muscle and nerve cells. Attempts are generally made to improve the oxygen supply in the human body through sports, good nutrition or medicine. As a rule, however, the oxygen supply of each individual can still be improved, if necessary with the help of nutritional supplements.

Oxidative stress, i.e. the formation of reactive oxygen species in the body, is considered potentially disease-causing and it is considered desirable to reduce oxidative stress. Nitric oxide (NO) acts as a radical scavenger, which can scavenge reactive oxygen species, and as a signal transmitter. A slightly increased concentration of endogenous nitric oxide is considered positive.

Accordingly, as a nutritional supplement, it would be desirable to have a powdered composition that dissolves as quickly and completely as possible in water to provide a healthy, drinkable solution, wherein the solution has an appealing appearance and good taste, enables high bioavailability of the ingredients, improves oxygenation in the human body, reduces oxidative stress, and slightly increases the concentration of endogenous nitric oxide.

The present disclosure is directed to these objectives and solves at least part of the tasks by a powdered composition for the preparation of a solution (as a nutritional supplement) for oral administration, wherein the composition contains, per 100 g, 1 g to 85 g of at least one monosaccharide, 0.18 g to 0.70 g of at least one steviol glycoside, 1.5 g to 10 g of citric acid, 0.3 g to 3 g of vitamin C, at least two other vitamins, and an extract from green tea (Camellia sinensis).

A solution can be prepared by merely adding water to the powdered composition in a drinking vessel and stirring briefly at room temperature (about 20° C.). A suspension is considered a solution and is included in the disclosure as long as the suspended portions are substantially drunk with it and do not remain at the bottom of the drinking vessel. Compositions in which substantial components sediment to the bottom of the drinking vessel are not considered a solution within the meaning of the disclosure.

The advantage is that the components from the solution can enter the bloodstream particularly quickly.

The at least one monosaccharide leads to the rapid dissolution of the powder in water and good taste. The at least one steviol glycoside also contributes to the good taste. By combining at least one monosaccharide and at least one steviol glycoside, a particularly healthy product is obtained, since the amount of monosaccharides can be reduced and the solution still contains at least one monosaccharide as an energy carrier. The citric acid also contributes to the special taste experience. Artificial sweeteners are not required and preferably the composition does not contain artificial sweeteners.

The composition is surprisingly microbially stable. Artificial preservatives are not required and preferably the composition does not contain artificial preservatives.

The vitamin C and the at least two other vitamins are vitamins that are not part of the green tea extract. Preferably, the vitamin C and the at least two other vitamins are added as a pure substance.

The verbs “contain” and “comprise” and their conjugations also include the verb “consist of” and its conjugations.

Preferred embodiments of the disclosure may be combined with each other as long as the context does not indicate otherwise.

Preferred embodiments are also given in the claims.

In preferred embodiments, the composition contains, per 100 g, 5 g to 80 g of the at least one monosaccharide, 0.19 g to 0.60 g of the at least one steviol glycoside, 2.5 g to 7.0 g of citric acid, and 0.4 g to 2.5 g of vitamin C.

Preferably, the composition contains, per 100 g, 4.14 mg to 3000 mg of the extract from green tea (Camellia sinensis), preferably from leaves of green tea. The extract contains valuable polyphenols, catechins and contributes to the special taste of the solution.

Preferably the composition contains

a concentrate of broccoli (Brassica oleracea italica), in particular of seeds, sprouts and/or shoots of broccoli, and/or

a concentrate of broccoli (Brassica oleracea italica), in particular from florets and/or stems of broccoli, and/or

an extract from onion (Allium cepa, in particular Allium cepa alliaceae), in particular from the bulb of the onion, and/or

an extract from apple (Malus domestica), in particular from the apple fruit, in particular from peels of the apple fruit, and/or

an extract from acerola (Malpighia glabra, in particular Malpighia glabra linne), in particular from the fruit of acerola, and/or

a concentrate of tomato (Lycopersicon esculentum), in particular from the fruit of the tomato, and/or

a concentrate of turmeric (Curcuma longa), in particular from the root of turmeric, and/or

a concentrate of garlic (Allium sativum), in particular from the garlic clove, and/or

a concentrate of basil (Ocimum basilicum), in particular of basil leaves, and/or

a concentrate of oregano (Origanum vulgare), in particular of leaves of oregano, and/or

a concentrate of cinnamon tree (Cinnamonum cassia), in particular from the bark (without cork) of the cinnamon tree, and/or

a concentrate of carrot (Dacus carota, in particular Dacus carota sativa), in particular from the root of the carrot, and/or

a concentrate of elderberry (Sambucus nigra), in particular from the fruit of the elderberry, and/or

an extract from currant (Ribes), in particular of the black currant (Ribes nigrum), in particular of the fruit of the black currant, and/or

a concentrate of blueberry (Vaccinium), in particular from the fruit of the blueberry, and/or

a concentrate of raspberry (Rubus idaeus), in particular of the fruit of the raspberry, and/or

a concentrate of blackberry (Rubus spp.), in particular from the fruit of the blackberry, and/or

a concentrate of chokeberry (Aronia, in particular Aronia melanocarpa), especially from the fruit of the chokeberry, and/or

a concentrate of spinach (Spinacia oleracea), in particular from the leaves of the spinach, and/or

a concentrate of cherry (Prunus avium), in particular from the cherry fruit, and/or

a concentrate of Brussels sprouts (Brassica oleracea gemmifera), especially florets of Brussels sprouts.

Preferably, the composition contains a total of 0.10 g to 0.75 g of these extracts and/or concentrates per 100 g of the composition.

Preferably the composition contains

a concentrate of broccoli (Brassica oleracea italica), in particular of seeds, sprouts and/or shoots of broccoli, and

a concentrate of broccoli (Brassica oleracea italica), in particular florets and/or stems of broccoli, and

an extract from onion (Allium cepa, in particular Allium cepa alliaceae), in particular the bulb of the onion, and

an extract from apple (Malus domestica), in particular of apple fruit, in particular of apple fruit peel, and

an extract from acerola (Malpighia glabra, in particular Malpighia glabra linne), in particular the fruit of acerola, and

a concentrate of tomato (Lycopersicon esculentum), in particular from the fruit of the tomato, and

a concentrate of turmeric (Curcuma longa), in particular from the root of turmeric, and

a concentrate of garlic (Allium sativum), especially from the garlic clove, and

a concentrate of basil (Ocimum basilicum), in particular of basil leaves, and

a concentrate of oregano (Origanum vulgare), especially leaves of oregano, and

a concentrate of cinnamon tree (Cinnamonum cassia), in particular from the bark (without cork) of the cinnamon tree, and

a concentrate of carrot (Dacus carota, in particular Dacus carota sativa), especially the root of the carrot, and

a concentrate of elderberry (Sambucus nigra), in particular from the fruit of the elderberry, and

an extract from blackcurrant (Ribes), in particular of blackcurrant (Ribes nigrum), in particular of the fruit of the blackcurrant, and

a concentrate of blueberry (Vaccinium), in particular from the fruit of the blueberry, and

a concentrate of raspberry (Rubus idaeus), in particular from the fruit of the raspberry, and

a concentrate of blackberry (Rubus spp.), especially the fruit of the blackberry, and

a concentrate of chokeberry (Aronia, in particular Aronia melanocarpa), especially the fruit of the chokeberry, and

a concentrate of spinach (Spinacia oleracea), in particular from the leaves of the spinach, and

a concentrate of cherry (Prunus avium), in particular from the cherry fruit, and

a concentrate of Brussels sprouts (Brassica oleracea gemmifera), especially florets of Brussels sprouts.

Preferably, the composition contains 0.10 g to 0.75 g total of these extracts and concentrates per 100 g of the composition.

The term “extract” means a dried-up extract from corresponding plant parts, wherein the extract is preferably obtained by means of an organic solvent and/or water and with a drug-extract ratio between 120:1 and 10:1. A “concentrate” means a dehydrated product from corresponding plant parts. Extracts and concentrates from fruits, leaves, bulbs, bark (excluding cork), roots, rhizomes, tubers, seeds, sprouts, shoots, florets, and stems provide high levels of valuable plant secondary metabolites such as polyphenols, depending on the plant species. Polyphenols are aromatic compounds that contain two or more hydroxyl groups directly attached to an aromatic ring and are classified as plant secondary metabolites. Polyphenols exhibit various health-promoting effects; in particular, some polyphenols have antioxidant effects. The concentrates and extracts of a wide variety of plants provide a variety of health-promoting plant secondary metabolites, vitamins, and polyphenols. Such a composition may increase the bioavailability of the compounds contained in the composition. Therefore, the result is a composition that has a particularly health-promoting effect and is particularly supportive of a balanced diet. Furthermore, the inventors have discovered that the addition of the extracts and/or concentrates mentioned in the patent application has a positive effect on the dissolving behavior of the powder in water and the taste of the solution.

Preferably, the composition contains per 100 g in each case 4 mg to 200 mg, preferably 5 mg to 50 mg, of one or more ingredients selected from an extract from pepper (Piper nigrum) and an extract from ginger (Zingiber officinale). Preferably, the composition contains, per 100 g, 4 mg to 200 mg, preferably 5 mg to 50 mg, of an extract from pepper (Piper nigrum), 0.25 mg to 2 g, preferably 0.45 mg to 1.5 g, of a curcumin compound, and 4 mg to 200 mg, preferably 5 mg to 50 mg, of an extract from ginger (Zingiber officinale). The curcumin compound may be a curcumin-cyclodextrin complex, particularly a curcumin-gamma-cyclodextrin complex. Preferably, the extract from ginger is an extract from the rhizome of ginger (Zingiber officinale). These substances, even in such small doses, act as bioenhancers and increase the bioavailability of the ingredients of the composition.

Preferably, the composition contains, per 100 g, 2.3 mg to 5 g, more preferably 4.14 mg to 3 g, of extract from coffee (Coffea arabica), preferably from the bean of the coffee.

Preferably, the composition contains, per 100 g, 0.025 mg to 2 g, more preferably 0.045 mg to 1.5 g, of sour cherry (Prunus cerasus) extract, preferably from the cherry of the sour cherry.

Preferably, the composition contains, per 100 g, 0.025 mg to 2 g, more preferably 0.045 mg to 1.5 g, of extract from cabbage (Brassica oleracea acephala), preferably from leaves of cabbage.

Preferably, the composition contains, per 100 g, 0.25 mg to 2 g, more preferably 0.45 mg to 1.5 g, of extract from turmeric (Curcuma longa), preferably from the root of turmeric.

Preferably, the composition contains, per 100 g, 0.025 mg to 2 g, more preferably 0.045 mg to 1.5 g, of extract from broccoli (Brassica oleracea italica), preferably from the floret of broccoli.

Preferably, the composition contains, per 100 g, 0.025 mg to 2 g, more preferably 0.045 mg to 1.5 g, of extract from blueberry (Vaccinium corymbosum), preferably from the fruit of the blueberry.

Preferably, the composition contains an extract from coffee (Coffea arabica), preferably from the bean of the coffee, an extract from sour cherry (Prunus cerasus), preferably from the cherry of the sour cherry, an extract from cabbage (Brassica oleracea acephala), preferably from leaves of the cabbage, an extract from turmeric (Curcuma longa), preferably from the root of the turmeric, an extract from broccoli (Brassica oleracea italica), preferably from the floret of broccoli, an extract from blueberry (Vaccinium corymbosum), preferably from the fruit of blueberry, and the extract from green tea (Camellia sinensis), preferably from leaves of green tea, wherein the composition contains, per 100 g, 0.01 g to 5 g in total of these extracts. These extracts, especially in combination, can increase the concentration of endogenous nitric oxide.

Preferably, the composition contains 100 mg to 1000 mg of caffeine per 100 g of the composition. The caffeine may be derived from a guarana extract.

In one embodiment of the disclosure, the at least two further vitamins are selected from B vitamins (B vitamin embodiment). Preferably, the composition in this embodiment contains 1 g to 85 g, more preferably 5 to 80 g, of the at least one monosaccharide per 100 g of the composition. Preferably, the composition in this embodiment contains 0.19 g to 0.60 g of the at least one steviol glycoside per 100 g of the composition. Preferably, the composition in this embodiment contains 2.5 g to 7.0 g of citric acid per 100 g of the composition. Preferably, the composition in this embodiment further contains, 50 mg to 200 mg of algae powder and optionally 3 g to 12 g, more preferably 4 g to 10 g, of guarana extract per 100 g of the composition. The guarana extract may contribute, among other things, caffeine. Preferably, the algae powder is a powder from brown algae. Preferably, in this embodiment, the composition contains dextrose as the at least one monosaccharide. In this embodiment, per 100 g of the composition, the composition preferably contains as B vitamins 100 mg to 1500 mg niacin, 20 mg to 270 mg pantothenic acid, 5 mg to 100 mg pyridoxine, 4 mg to 72 mg riboflavin, 5 mg to 70 mg thiamine, 10 pg to 10 mg cyanocobolamine, 0.5 mg to 7 mg biotin, and 0.5 mg to 12 mg folic acid. Dextrose dissolves very quickly in water and, in combination with the other ingredients, gives the solution its special taste. Furthermore, dextrose improves the absorption of certain ingredients. In particular, this embodiment helps to improve the oxygen supply to the tissues.

In an alternative embodiment of the disclosure, the at least two further vitamins are selected from vitamins A and E (vitamins A and E embodiment). Preferably, the composition in this embodiment contains 1500 μg to 4000 μg of retinol equivalents and 60 mg to 120 mg of α-tocopherol equivalents per 100 g of the composition. Preferably, the composition in this embodiment contains 5 g to 80 g, more preferably 5 g to 70 g, most preferably 5 g to 42 g, of the at least one monosaccharide per 100 g of the composition. Preferably, the composition in this embodiment contains 0.19 g to 0.60 g of the at least one steviol glycoside per 100 g of the composition. Preferably, the composition in this embodiment contains 2.0 g to 7.0 g of citric acid per 100 g of the composition. Preferably, the composition in this embodiment comprises fructose as the at least one monosaccharide. Preferably, the composition in this embodiment contains 200 μg to 350 μg of selenium per 100 g of the composition. Preferably, the composition in this embodiment contains 35 g to 85 g of dietary fiber per 100 g of the composition. The dietary fibers are preferably vegetable fibers from oats, pea, rice and/or apple. Fructose dissolves very quickly in water and, in combination with the other ingredients, gives the solution its beneficial taste. It is amazing that this particular composition can be used to produce a solution despite the high fiber content. This embodiment is particularly beneficial for intestinal health and stimulates the immune system. The ingredients of the composition can be absorbed even better in this variant. In this alternative embodiment, it is also preferred that the composition contains digestive enzymes (amylase, protease, lactase, cellulase and lipase) and a curcumin-gamma-cyclodextrin complex. In this alternative embodiment, it is also preferred that the composition contains gram-positive non-sporulating bacteria, such as (Lactobacillus acidophilus, Lactobacillus reuteri) and/or gram-positive sporulating bacteria, such as Bacillus species.

The composition may further contain excipients such as beet powder (concentrate of beet juice, maltodextrin), gum arabic, pectin and/or guar gum.

The disclosure also relates to the use of a composition according to the disclosure for the preparation of a solution for oral application—or in other word—a method for the preparation of a solution for oral application. Here, the powdered composition is dissolved in water to produce the solution.

In this regard, in the B vitamin embodiment, 1.25 g to 2.25 g of the composition is added to approximately 40 mL of water at room temperature and mixed to obtain a solution. The product is preferably consumed three times daily.

In the vitamins A and E embodiment, 8 g to 16 g of the composition is added to approximately 180 mL of water at room temperature and mixed to obtain a solution. The product is preferably consumed once a day.

The disclosure is explained below by means of two examples:

Example formulation for the B vitamin embodiment

100 g of the powdered composition of this example contain:

Vitamin C, 2.5 g;

Niacin, 0.34 g;

Caffeine, 0.8 g;

Pantothenic acid, 0.05 g;

Pyridoxine, 0.02 g;

Thiamine, 0.02 g;

Riboflavin, 0.02 mg;

Cyanocobolamine, 10 μg;

Folic acid, 2.0 mg;

Biotin, 1.5 mg;

Citric acid, 7.5 g;

Guarana extract, 9.5 g;

Steviol glycosides, 0.4 g;

Algae powder, 0.1 g;

Green tea extract (Camellia sinensis), 5 mg;

Beet powder (concentrate of beet juice, maltodextrin), 1 g;

Dextrose ad 100 g.

Example formulation for vitamins A and E embodiment

100 g of the powdered composition of this example contain:

Steviol glycosides, 0.3 g;

Citric acid, 3.5 g;

Vitamin C, 1.5 g;

Green tea extract (Camellia sinensis), 0.08 g;

2000 μg retinol equivalents;

70 mg α-tocopherol equivalents;

250 μg selenium;

46 g dietary fiber;

Lactobacillus acidophilus, Lactobacillus reuteri, 0.3 g;

Amylase, protease, lactase, cellulase and lipase, 0.3 g;

Fructose ad 100 g.

The powdered compositions dissolve quickly in water and form healthy, drinkable solutions without artificial preservatives or sweeteners. The solutions have an attractive appearance and good taste, provide high bioavailability of ingredients and improve the supply of oxygen, essential vitamins, polyphenols and other substances to the human body.

1. Powder composition for the preparation of a solution for oral administration, wherein
the composition contains, per 100 g,
1 g to 85 g of at least one monosaccharide,
0.18 g to 0.70 g of at least one steviol glycoside,
1.5 g to 10 g citric acid,
0.3 g to 3 g vitamin C,
at least two other vitamins,
an extract from green tea (Camellia sinensis),
4 mg to 200 mg of an extract from pepper (Piper nigrum),
0.25 mg to 2 g of a curcumin compound, and
4 mg to 200 mg of an extract from ginger (Zingiber officinale).

2. (canceled)

3. Composition according to claim 1, wherein
the composition contains, per 100 g, 2.3 mg to 5 g of extract from coffee (Coffea arabica).

4. Composition according to claim 1, wherein
the composition contains, per 100 g, 0.025 mg to 2 g of extract from broccoli (Brassica oleracea italica).

5. Composition according to claim 1, wherein
the composition contains an extract from coffee (Coffea arabica), an extract from sour cherry (Prunus cerasus), an extract from cabbage (Brassica oleracea acephala), an extract from turmeric (Curcuma longa), an extract from broccoli (Brassica oleracea italica) and an extract from blueberry (Vaccinium corymbosum).

6. Composition according to claim 1, wherein
the at least two other vitamins are selected from B vitamins, and the composition contains, per 100 g of the composition, and 50 mg to 200 mg of algae powder.

7. Composition according to claim 6, wherein
the at least one monosaccharide is dextrose.

8. Composition according to claim 6, wherein the composition contains as B vitamins, per 100 g of the composition,
100 mg to 1500 mg niacin,
20 mg to 270 mg pantothenic acid,
5 mg to 100 mg pyridoxine,
4 mg to 72 mg riboflavin,
5 mg to 70 mg thiamine,
10 μg to 10 mg cyanocobolamine,
0.5 mg to 7 mg biotin and
0.5 to 10 mg folic acid.

9. Composition according to claim 1, wherein
the composition contains 100 mg to 1000 mg of caffeine per 100 g of the composition.

10. Composition according to claim 1, wherein
the at least two other vitamins are selected from vitamins A and E and the composition contains, per 100 g of the composition, 1500 μg to 4000 μg of retinol equivalents and 60 mg to 120 mg of a-tocopherol equivalents.

11. Composition according to claim 10, wherein
the at least one monosaccharide is fructose.

12. Composition according to claim 10, wherein
the composition contains 200 μg to 350 μg of selenium per 100 g of the composition.

13. Composition according to claim 10, wherein
the composition contains 35 g to 85 g of dietary fiber per 100 g of the composition.

14. Composition according to claim 10, wherein
the composition contains
i) amylase, protease, lactase, cellulase and lipase, and
(ii) a curcumin-gamma-cyclodextrin complex.

15. Use of a composition according to claim 1 for the preparation of a solution for oral administration.

16. The composition according to claim 1, wherein the composition contains a concentrate of broccoli (Brassica oleracea italica).

17. Composition according to claim 6, wherein the composition further contains, per 100 g of the composition, 3 g to 12 g of guarana extract.

18. Composition according to claim 14, wherein the composition further contains gram-positive non-sporulating bacteria and/or gram-positive sporulating bacteria.

19. Composition according to claim 18, wherein the gram-positive non-sporulating bacteria is Lactobacillus acidophilus and/or Lactobacillus reuteri.

20. Composition according to claim 18, wherein gram-positive sporulating bacteria is Bacillus species.
